(19) United States
(12) Patent Application Publication
(10) Pub. No.: US 2026/0014149 A1
(43) Pub. Date: Jan. 15, 2026

(54) METHODS OF TREATING MERKEL CELL CARCINOMA WITH TRYPTOPHAN HYDOXYLASE INHIBITORS

(71) Applicant: TerSera Therapeutics LLC, Deerfield, IL (US)

(72) Inventors: Nancy Elizabeth Martin, Deerfield, IL (US); Lonnie Don Brent, Deerfield, IL (US)

(73) Assignee: TerSera Therapeutics LLC, Deerfield, IL (US)

(21) Appl. No.: 18/772,686

(22) Filed: Jul. 15, 2024

(51) Int. Cl. A61K 31/506; A61K 39/395

(52) U.S. Cl. CPC A61K 31/506; A61K 39/3955

(57) ABSTRACT

Described herein are methods of treating a patient with Merkel cell carcinoma by administering a tryptophan hydroxylase inhibitor such as telotristat to the patient. In an aspect, the patient has functional Merkel cell carcinoma, that is, Merkel cell carcinoma that secretes serotonin. The tryptophan hydroxylase inhibitor may be orally administered or applied topically to the site of the lesion.

### FIELD OF THE DISCLOSURE

The present disclosure is related to the use of tryptophan hydroxylase inhibitors to treat Merkel cell carcinoma.

### BACKGROUND

Merkel cell carcinoma, also called neuroendocrine carcinoma of the skin, is a rare, aggressive skin cancer that typically appears in sites exposed to sunlight, typically as rapidly growing purple-red nodules. Age, light skin color, and a weakened immune system increase the risk of developing Merkel cell carcinoma. Merkel cell carcinoma not only grows quickly, but also spreads quickly, often metastasizing to other parts of the body. Merkel cell carcinoma has a very poor clinical prognosis and is one of the most lethal tumors of cutaneous origin. Treatments include surgical resection, radiation and chemotherapy, although the recurrence rate is as high as 40%. More recently, with the rise and wide application of immune checkpoint inhibitors (PD-L/PD-L1 and CTLA-4 inhibitors), some monoclonal antibodies have been applied to the treatment of advanced metastatic Merkel cell carcinoma, for example. However, this treatment method relies heavily on the autoimmunity of patients, and the response rate varies greatly for different individuals.

What is needed are new treatments for Merkel cell carcinoma.

### BRIEF SUMMARY

In an aspect, a method of treating a patient with Merkel cell carcinoma comprises administering a tryptophan hydroxylase inhibitor to the patient.

In another aspect, the Merkel cell carcinoma is a functional Merkel cell carcinoma.

### DETAILED DESCRIPTION

Tryptophan hydroxylase (TPH) converts tryptophan to 5-hydroxytryptophan, which is subsequently converted to serotonin. Serotinin is secreted by cells and then metabolized to 5-hydyroxyindoleacetic acid (5-HIAA), a biomarker measurable in both urine and plasma. Further, some functional neuroendocrine neoplasms, those that secrete serotonin, have been shown to have increased expression of tryptophan hydroxylase. Administration of tryptophan hydroxylase inhibitors such as telotristat etiprate reduces serotonin production as evidenced by decreased urinary or plasma 5-HIAA.

Described herein is the use of tryptophan hydroxylase inhibitors to treat patients, e.g., human patients, with Merkel cell carcinoma, a neuroendocrine carcinoma of the skin.

The term “administering” or “administration,” as used herein, refers to providing the compound or pharmaceutical composition of the invention to a subject suffering from or at risk of the diseases or conditions to be treated or prevented.

A “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Merkel cells are neuroendocrine cells within the epidermis that are involved in mechanoreception. Merkel cells express epithelial and neuroendocrine markers including serotonin. Release of serotonin from Merkel cells is integral to electrophysiological and behavioral responses to tactile stimulation. The mRNA for tryptophan hydroxylase is expressed in Merkel cells (Chang et al., “Merkel disc is a serotonergic synapse in the epidermis for transmitting tactile signals in mammals”, PNAS, 113 (37) E5491-E5500 (2016)).

The cause of Merkel cell carcinoma is unknown, but it has been associated with Merkel cell polyomavirus. In addition, UV damage to DNA as well as a weakened immune system are associated with Merkel cell carcinoma.

In an aspect, the Merkel cell carcinoma is a functional Merkel cell carcinoma, that is, a Merkel cell carcinoma that secretes serotonin.

Merkel cell carcinoma can be divided into stages as follows:

Stage 0: Carcinoma in situ, abnormal Merkel cells found in top layer of skin. No spread to lymph nodes, or distant metastasis.

Stage I: Tumor is 2 cm or smaller. No spread to lymph nodes, or distant metastasis.

Stage IIA: Tumor is larger than 2 cm. No spread to lymph nodes, or distant metastasis.

Stage IIB: Tumor has spread to nearby tissue such as cartilage, muscle or bone. No spread to lymph nodes, or distant metastasis.

Stage IIIA: Any size tumor with spread to nearby tissue and cancer found in lymph node by biopsy, but not seen on physical exam or imaging. No distant metastasis. Alternatively, no sign of primary cancer, but swollen lymph node found on physical exam or by imaging. Cancer found in lymph node. No distant metastasis.

Stage IIIB: Any size tumor, with spread to nearby tissue or bone. Cancer found in lymph node by biopsy. No distant metastasis. Alternatively, cancer in a lymph vessel between primary tumor and far away lymph nodes. Cancer may have spread to lymph nodes. No distant metastasis.

Stage IV: Metastatic disease. Cancer has spread to skin a distance from primary tumor, or spread to other parts of the body such as the liver, lung, bone or brain.

In an aspect, the Merkel cell carcinoma is stage 0, I, II or III, preferably stage I or II. Without being held to theory, it is believed that administration of a tryptophan hydroxylase inhibitor during the early stages of Merkel cell carcinoma, particularly stages 0 to II will slow progression to later stages, such as progression to nearly tissues, lymph nodes and/or distant sites, i.e., metastasis.

In another aspect, the Merkel cell carcinoma is characterized by a lesion at the primary site.

In another aspect, the Merkel cell carcinoma is metastatic Merkel cell carcinoma, e.g., stage IV Merkel cell carcinoma. It is hypothesized that tryptophan hydroxylase inhibitors will reduce tumor burden reduction. Tumor burden, also called tumor load, includes the number of cancer cells, the size of a tumor, and/or the amount of cancer in the body. It is believed that serotonin propagates tumors in Merkel cell carcinoma, so by administering a tryptophan hydroxylase inhibitor, the serotonin production will be reduced, thus decreasing tumor burden. In an aspect, the tryptophan hydroxylase inhibitor is coadministered with an immunotherapy.

In an aspect, the patient's Merkel cell carcinoma is diagnosed by a skin biopsy. Merkel cell carcinoma typically manifests as a firm, flesh colored or red nodule or plaque. The lesion usually increases in size rapidly, often over a period of weeks or months. Because Merkel cell carcinomas can be confused with other malignant and benign tumors, a skin biopsy can be important for proper diagnosis. The skin biopsy can be a punch, incisional, or excisional biopsy. Immunohistochemistry to confirm the expression of epithelial markers such as cytokeratin 20 (CK-20) and/or neuro-endocrine markers such as neuron-specific enolase (very sensitive but expressed by other neuroendocrine tumors), synaptophysin, CD56 and chromogranin A (more specific for Merkel cell carcinoma) can be employed. Merkel cell carcinoma cells are negative for thyroid transcription factor 1 (TTF-1), S100B, and leukocyte-common antigen (LCA). In general, a positive CK-20 and negative TTF-1 immunostaining is usually considered sufficient for the diagnosis of Merkel cell carcinoma.

In an aspect, after diagnosis with Merkel cell carcinoma, sentinel lymph node evaluation is performed. Sentinel lymph nodes are the first lymph nodes to receive lymphatic drainage from the primary tumor and the first lymph node the cancer is likely to spread to. Sentinel lymph nodes are generally removed during an excisional surgical procedure. To identify sentinel lymph nodes, a radioactive tracer or a blue dye is injected near the tumor and the first lymph node to receive the tracer or dye is removed and biopsied.

Exemplary patients for the methods described herein are human patients.

In an aspect, the human patient is an immunosuppressed patient, such as a patient with a hematological malignancy (non-Hodgkin lymphoma, chronic lymphocytic leukemia), human immunodeficiency virus (HIV) infection/acquired immune deficiency syndrome (AIDS), an immune-mediated inflammatory disease (inflammatory bowel disease, rheumatoid arthritis), a recipient of a solid organ transplant, and the like.

In an aspect, the patient is white and over the age of 50.

The methods described herein include administering a tryptophan hydroxylase inhibitor to a patient with Merkel cell carcinoma. Tryptophan hydroxylase inhibitors include telotristat, rodatristat, xanthine-imidazopyridine and imidazothiazole TPH inhibitors (e.g., TPH-004) and xanthine-benzimidazole derivatives (TPG-0001), described in U.S. Pat. Nos. 10,214,530 and 10,683,309, and the inhibitors described in U.S. Pat. No. 7,553,840 and the inhibitors described in U.S. Pat. No. 9,199,994. U.S. Pat. Nos. 7,553,840, 9,199,994, 10,214,530 and 10,683,309 are incorporated by reference herein for their disclosure of tryptophan hydroxylase inhibitors.

Telotristat ethyl ((S)-ethyl 2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl) propanoate; TE) is commercially available as XERMELO®, which is US FDA approved for the treatment of carcinoid syndrome diarrhea in combination with somatostatin analog (SSA) therapy in adults inadequately controlled by SSA therapy. TE is a tryptophan hydroxylase inhibitor and telotristat, the active metabolite, mediates the rate-limiting step in serotonin biosynthesis. By inhibiting tryptophan hydroxylase, TE and its metabolite reduce the production of peripheral serotonin and also the frequency of carcinoid syndrome diarrhea.

As used herein, telotristat can be administered as telotristat etiprate, which undergoes hydrolysis to the active compound telotristat. Telotristat ethyl can be in the form of a salt such as telotristat etiprate. Telotristat etiprate is the hippurate salt of telotristat ethyl [(S)-ethyl 2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl) propanoate], which undergoes hydrolysis to the active metabolite, (S)-2-amino-3-(4-(2-amino-6-((R)-1-(4-chloro-2-(3-methyl-1H-pyrazol-1-yl)phenyl)-2,2,2-trifluoroethoxy)pyrimidin-4-yl)phenyl) propanoic acid. The molecular formula of telotristat etiprate is C₂₇H₂₆ClF₃N₆O₃·C₉H₉NO₃ and its molecular weight is 754.2. The molecular weight of the free base (telotristat ethyl) is 575.0.

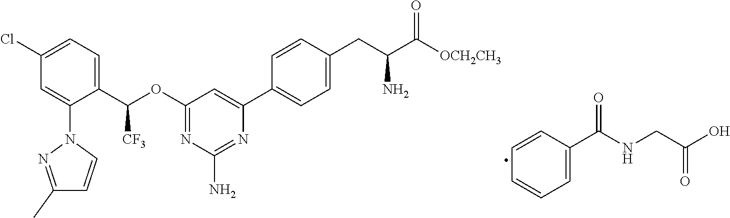 [SMILES: CCOC(=O)[C@@H](N)Cc1ccc(-c2cc(O[C@H](c3ccc(Cl)cc3-n3ccc(C)n3)C(F)(F)F)nc(N)n2)cc1 ; O=C(O)CNC(=O)c1ccccc1]

As used herein, salts may be prepared from pharmaceutically acceptable non-toxic acids or bases including inorganic acids and bases and organic acids and bases. Exemplary pharmaceutically acceptable salts include hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnarnate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate and the like.

Exemplary doses of telotristat ethyl for oral administration, for example are up to 1500 mg/day, or preferably 250 mg three times daily, for a total daily dose of 750 mg. Exemplary dosage strengths of telotristat ethyl for topical administration, for example are 0.01 to 10 wt %.

The tryptophan hydroxylase inhibitor may be administered in a form for the treatment of Merkel cell carcinoma such as orally, topically, and the like. Pharmaceutical compositions include a tryptophan hydroxylase inhibitor and a pharmaceutically acceptable excipient.

In some embodiments, the pharmaceutical compositions can be prepared as solid dosage forms for oral administration (e.g., capsules, tablets, pills, dragees, powders, granules, and the like). A tablet can be prepared by compression or molding. Compressed tablets can include one or more binders, lubricants, glidants, inert diluents, preservatives, disintegrants, or dispersing agents. Tablets and other solid dosage forms, such as capsules, pills and granules, can include coatings, such as enteric coatings.

Dosage forms for transdermal administration include powders, sprays, ointments, pastes, creams, lotions, gels, solutions, and patches. Gel formulations can include, for example, cellulose derivatives, carbomers, alginates, gum tragacanth, gelatin, pectin, carrageenan, gellan gum, starch, Xanthan gum, guar gum, agar, non-cellulosic polysaccharides, vinyl polymers, acrylic resins, polyvinyl alcohol, carboxyvinyl polymers, hyaluronic acid, and the like. Lotions can include cellulose derivatives, glycerin, non-cellulosic polysaccharides, polyethylene glycols of different molecular weights, propanediol, and the like. Pastes or ointments can include glycerin, petroleum jelly, paraffin, polyethylene glycols of different molecular weights, and the like. Creams or foams can include hydroxypropyl methyl cellulose, gelatin, polyethylene glycols of different molecular weights, sodium dodecyl sulfate, sodium fatty alcohol polyoxyethylene ether sulfonate, gluten powder, acrylamide, and the like. Powder aerosols can include mannitol, glycine, dextrin, dextrose, sucrose, lactose, sorbitol, polysorbates, and the like. Liquid aerosol sprays can include viscosity modifiers, such as hyaluronic acid, emulsifiers, buffering agents, and the like.

Moisturizing agents, such as glycerol, glycerin, polyethylene glycol, trehalose, petrolatum, paraffin oil, hyaluronic acid and salts (e.g., sodium and potassium salts) thereof, octanoic/caprylic triglyceride, and the like; and/or antioxidants, such as vitamins and glutathione; and/or pH modifiers, such as acids, bases and pH buffers, may also be included in topical formulations. Surfactants/emulsifiers, such as hexadecanol (cetyl alcohol), fatty acids (e.g., stearic acid), sodium dodecyl sulfate (sodium lauryl sulfate), sorbitan esters (e.g. sorbitan stearate, sorbitan oleate, etc.), monoacyl glycerides (such as glyceryl monostearate) polyethoxylated alcohols, polyvinyl alcohols, polyol esters, polyoxyethylene alkyl ethers (e.g., polyoxyethylene sorbitan monooleate), polyoxyethylene castor oil derivatives, ethoxylated fatty acid esters, polyoxylglycerides, lauryl dimethyl amine oxide, bile salts (e.g. sodium deoxycholate, sodium cholate), phospholipids, N,N-dimethyldodecylamine-N-oxide, hexadecyltrimethyl-ammonium bromide, poloxamers, lecithin, sterols (e.g., cholesterol), sugar esters, polysorbates, and the like; preservatives, such as phenoxyethanol, ethylhexyl glycerin, and the like; and thickeners, such as acryloyldimethyltaurate/VP copolymer may be included.

In an aspect, administration of the tryptophan hydroxylase inhibitor is combined with another therapy for Merkel cell carcinoma such as surgical excision, radiation therapy, immunotherapy, chemotherapy, and the like.

In the treatment of Merkel cell carcinoma, surgical excision is generally the first therapeutic intervention. Margins of 1 cm to 2 cm may be recommended. Wide local excision as well as Mohs micrographic surgery can be employed. The tryptophan hydroxylase inhibitor may be administered before or after surgical excision of the Merkel cell carcinoma. In an aspect, when the tryptophan hydroxylase inhibitor is administered after surgical excision it reduces the risk of recurrence in the patient. The risk of recurrence after surgery without administering a tryptophan hydroxylase inhibitor can be as high as 50%. In an aspect, when the tryptophan hydroxylase inhibitor is administered after surgical excision it increases the time to recurrence, which can be within 2-3 years after surgical excision without administering a tryptophan hydroxylase inhibitor.

Radiation therapy can be employed after surgical excision to improve local and regional relapse-free survival, disease-free survival, metastasis-free survival and/or overall survival. Adjuvant radiation therapy can be performed within 8 weeks of excision surgery. Lymph node irradiation may also be employed, particularly in Stage III disease.

Alternatively, radiation therapy can be used as a therapy in patients for whom surgery is not an option, such as elderly patients for whom anesthesia and surgery are high risk. Radiation therapy can also be used for locoregional control of Merkel cell carcinoma.

Immunotherapy includes treatment with immune checkpoint inhibitors including anti-PD-L1 antibodies such as avelumab and durvalumab, anti-PD-1 antibodies such as nivolumab, retifanlimab-dlwr, and pembrolizumab, and anti-CTLA-4 antibodies such as ipilimumab and tremelimumab, and combinations thereof.

Chemotherapy for Merkel cell carcinoma includes cisplatin, carboplatin, etoposide, and combinations thereof.

The invention is further illustrated by the following non-limiting examples.

### Example

Telotristat ethyl will be tested in a proof-of-concept study, preferably a randomized, placebo-controlled, parallel group study in patients with diagnosed Merkel cell carcinoma, preferably metastatic Merkel cell carcinoma. Comparative groups can include placebo and/or best available therapy, typically immunotherapy (e.g., avelumab). A projected endpoint is tumor burden reduction at the end of 12 weeks. Response Evaluation Criteria in Solid Tumors (RECIST) may be used as a standard to determine progression and tumor response.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a patient with Merkel cell carcinoma, comprising administering a tryptophan hydroxylase inhibitor to the patient.

2. The method of claim 1, wherein the Merkel cell carcinoma is a functional Merkel cell carcinoma.

3. The method of claim 1, wherein the Merkel cell carcinoma is a stage 0, I, II or III Merkel cell carcinoma.

4. The method of claim 1, wherein the Merkel cell carcinoma is a stage 0, I, or II Merkel cell carcinoma.

5. The method of claim 3, wherein the Merkel cell carcinoma is characterized by a lesion at the primary site.

6. The method of claim 1, wherein the Merkel cell carcinoma is a stage 0, I, or II Merkel cell carcinoma.

7. The method of claim 6, wherein the Merkel cell carcinoma is characterized by a lesion at the primary site.

8. The method of claim 1, wherein the Merkel cell carcinoma is metastatic Merkel cell carcinoma.

9. The method of claim 8, wherein the tryptophan hydroxylase inhibitor reduces tumor burden in the patient.

10. The method of claim 8, wherein the tryptophan hydroxylase inhibitor is co-administered with an immunotherapy.

11. The method of claim 1, wherein the Merkel cell carcinoma is confirmed by skin biopsy.

12. The method of claim 1, wherein the tryptophan hydroxylase inhibitor is telotristat or a pharmaceutically acceptable salt thereof.

13. The method of claim 1, wherein the patient is a human patient.

14. The method of claim 13, wherein the patient is immunosuppressed.

15. The method of claim 14, wherein the immunosuppressed patient has a hematological malignancy, HIV infection, human immunodeficiency virus (HIV) infection, acquired immune deficiency syndrome (AIDS), an immune-mediated inflammatory disease or is a recipient of a solid organ transplant.

16. The method of claim 13, wherein the human patient is white and over the age of 50.

17. The method of claim 1, wherein the tryptophan hydroxylase inhibitor is orally administered.

18. The method of claim 1, wherein the tryptophan hydroxylase inhibitor is topically administered to the site of the Merkel cell carcinoma.

19. The method of claim 1, wherein the tryptophan hydroxylase inhibitor is administered before or after surgical excision of the Merkel cell carcinoma.

20. The method of claim 1, wherein the tryptophan hydroxylase inhibitor is administered after surgical excision of the Merkel cell carcinoma and the tryptophan hydroxylase inhibitor reduces the risk of recurrence in the patient, and/or reduces the time to recurrence in the patient.

21. The method of claim 1, wherein the method further comprises administering radiation therapy, immunotherapy, chemotherapy, or a combination thereof.

22. The method of claim 21, wherein the immunotherapy comprises an anti-PD-L1 antibody, and anti-PD-1 antibody, an anti-CTLA-4 antibody, or a combination thereof.
